(19) United States
(12) Patent Application Publication
(10) Pub. No.: US 2005/0170346 A1
(43) Pub. Date: Aug. 4, 2005

(54) Method and a kit for determination of a microbial count

(75) Inventors: Henrik Westh, Holte (DK); Gorm Lisby, Vekso (DK)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: QUANTIBACT A/S c/o Novi A/S, Copenhagen K (DK)

(21) Appl. No.: 10/498,998

(22) PCT Filed: Dec. 19, 2002
(86) PCT No.: PCT/DK02/00879

(30) Foreign Application Priority Data
Dec. 19, 2001 (DK) PA 2001 01920

(51) Int. Cl. C12Q 1/68

(52) U.S. Cl. 435/6

(57) ABSTRACT

The invention relates to the field of estimating a microbial count in a sample using molecular techniques. The invention furthermore relates to a kit for performing such estimation and use of the estimation method for providing bacterial cell counts in milk and for diagnosing infectious conditions in animals and human beings. Furthermore, the invention relates to a method for quality control of food (especially milk and dairy products), feed and water samples using molecular techniques. Reproducible and reliable estimates of a microbial count can be obtained using a molecular hybridisation assay between a target nucleic acid sequence and a labelled probe. Proper calibration and quantitative recording of the signal produced by the label can estimate a microbial count. One important feature of the invention is the use of locked nucleic acid (LNA) monomers in the probes.

This application is claims priority from Danish patent application No. PA 2001 01920 filed on 19 Dec. 2001, which is hereby incorporated by reference in its entirety. All patent and non-patent references cited in that application or in the present application are incorporated by reference in their entirety.

The invention relates to the field of estimating a microbial count in a sample using molecular techniques. The invention furthermore relates to a kit for performing such estimation and use of the estimation method for providing bacterial cell counts in milk and for diagnosing infectious conditions in animals and human beings. Furthermore, the invention relates to a method for quality control of food (especially milk and dairy products), feed and water samples using molecular techniques.

### PRIOR ART

Analysis of Milk Samples

Mastitis is an infectious conditions of the udder which is caused by bacteria such as Staphylococcus spp, in particular S. aureus, Streptococcus spp. and other bacteria. Most methods for detection of mastitis are based on the estimation of the number of somatic cells in the milk withdrawn from the udder. Determination of the number of somatic cells is an indirect measure of the infection and provides no information about the nature of the bacterial species causing the infection. In order to get more precise information relevant for selecting an appropriate treatment (antibiotic) it is of importance to know the species of bacterium causing the infection. Knowing the species is also important in order to be able to identify the source of infection.

In dairy agriculture somatic cell counts are used for quality control. Samples with somatic cell counts above a given (often authority decided) threshold are discarded because they are believed to arise from animals with mastitis.

Determinations or assessments of the number of somatic cells in milk have been performed by various methods. One of these methods is flow cytometry; instrument for performing flow cytometry is available, e.g., from Becton, Dickinson and Company, Franklin Lakes, USA.

Another known method for the determination of somatic cells in milk is based on the detection of signals from particles which are dispersed on the rim of a polished rotating disc, one such instrument available from Foss Electric, HillerØd, Denmark. The accuracy in the assessment of the number of particles using this method is dependent on the physical shape of the thin film of sample dispersed on the disk, and high sensitivity is needed to detect the weak signals from the particles in question in the course of the relative short period of time the particle is present in the detector.

One known method for the determination of somatic cells in milk based on spreading a film of milk onto a ribbon-like film which is then analysed by the means of a microscope, cf. European patent 0 683 395.

U.S. Pat. No. 5,722,343 and U.S. Pat. No. 4,385,590 relate to a combination of filtration of milk and the use of an optical sensor for detecting a contaminant including mastitis. Filtration is time consuming and the assessment of contaminants by means of an optical sensor does not represent an accurate and reliable assessment of milk quality. U.S. Pat. No. 3,668,925 relates to a method of filtration of milk, characterisation of leukocytes in the retained material, and correlation of a high number of leukocytes with the occurrence of an inflammatory condition such as mastitis.

The prior art also discloses that the electrical conductance or capacitance of a milk sample may be used as an indication of an inflammatory condition in the animal being milked. The technique is based on the alterations in conductance caused by somatic cells in the milk. However, alterations in electrical conductance or capacitance may have many other causes and often includes factors that are not related to the occurrence of an inflammatory condition such as mastitis. WO 95/22888 relates to the measurement of electrical conductance or capacitance, and the correlation of a certain value of an obtained result with the occurrence of mastitis. Correlation of electrical conductance or capacitance with an inflammatory condition including mastitis is also described in U.S. Pat. No. 5,873,323, U.S. Pat. No. 5,704,311, U.S. Pat. No. 5,664,521, and U.S. Pat. No. 4,771,007.

Further techniques for determining a somatic cell count in milk are based on analysing a relative large volume of milk in a microscope at low magnification and/or resolution and are described in WO 98/50777, WO 98/50577, WO 00/28297, WO 00/27183.

As can be seen from the above there are ample methods for rapid detection of the number of somatic cells in milk and there is a need in the art for rapid and reliable methods for determination of a direct bacterial count in milk samples.

Determination of Bacterial Counts

Methods for determination of a bacterial count in samples, in particular in urine samples are known in the art. The usual way to do this is to inoculate a small sample of the liquid or solid suspected of containing bacteria on a growth medium supporting the growth of bacteria and counting the number of colony forming units (CFU). One disadvantage of this method is that it is time consuming. The Petri plates with the bacteria need to be left for a specified period before a colony count can be performed. For urine samples the inoculation time on the growth medium is approximately 24 hours. Apart from this, the procedure requires manpower to perform the count. In the case of urine samples it is desirable to obtain the results much more rapidly, so that antibiotic treatment can be initiated as soon as possible and be avoided for patients without treatment-requiring infections.

Attempts have been made at developing methods for estimating a bacterial count using molecular probes, in particular probes based on conserved sequences in the 16S and/or 23S rRNA sequences.

U.S. Pat. No. 5,738,988 (Gen-Probe Inc) discloses a method for determining the presence of bacteria in a urine sample using labelled nucleotide probes which hybridise to a conserved region on 16S rRNA. It is documented that a probe based on sequences from Mycoplasma hominis can be used to recognise sequences in other Mollicutes, in E. coli, Legionella pneumoniae, Micrococcus, Streptococcus, Staphylococcus and Bacillus but not in yeast, humans and various animals. According to the specification it is possible to quantify the number of bacteria of a given species in a sample.

WO 90/15157 (Amoco) discloses a method for determination of the presence of bacteria in clinical and other samples using nucleotide probes based on 16S and 23S rRNA. Some of the probes appear to hybridise to sequences from several species and others only to one or very few species.

WO 91/00926 (Microprobe Corporation) discloses a method for determination of the number of bacteria in a sample based on a sandwich assay. The used sequences (complementary to 16S rRNA) hybridise to rRNA from Eubacteria but not to rRNA from eukaryots or archaebacteria. In one example the number of E. coli in a number of samples is determined and compared to results obtained by culture of the samples in a traditional assay. The data indicate that the dynamic range is restricted to 10⁵ to 10⁷ CFU per mL. A similar technical disclosure is given in EP 0 277 237 (Toray), where probes that distinguish Gram-positive from Gram-negative bacteria are used to determine the presence of the bacteria.

Whereas these disclosures claim that it is possible to obtain a bacterial count from a sample using the quantitative DNA based probes, no commercial products have so far been put on the market. This should be seen in view of the fact that the majority of the disclosures are more than 10 years old and in view of the fact that there is an enormous market for rapid determination of bacterial counts in clinical and other samples, such as food and water samples.

Apparently, there are problems associated with the technique, which were not realised at the time of drafting the above mentioned patent applications. One of the problems is illustrated in U.S. Pat. No. 5,738,988, where it is shown that the degree of hybridisation between the probe and the target sequence varies from 100 to almost nil depending on the bacterial species. This means that the occurrence of some bacteria in a mixed sample will be “under-estimated” or go undetected, whereas others may be “over-estimated” depending on the species used for calibration of the method. In real life, most samples do contain bacteria from multiple species and many samples (clinical, water, and food) are evaluated according to the total bacterial count irrespective of the bacterial species.

Another problem is that the sensitivity of the prior technique is not high enough. There is a need for determining a bacterial count at concentrations below 10⁴ CFU per mL and even down to 10² CFU per mL using molecular techniques. Such techniques are not disclosed in the prior art.

### SUMMARY OF THE INVENTION

According to a first aspect the invention relates to a method for determination of a microbial count in a sample comprising
i. providing a sample suspected of containing at least one species of micro-organism,
ii. lysing the micro-organisms present in the sample,
iii. contacting the sample with at least one nucleotide-probe comprising at least one locked nucleic acid (LNA) and being capable of selectively hybridising to a microbial target nucleic acid sequence,
iv. determining the amount of hybrid,
v. correlating the result to the number of at least one species of micro-organism in the sample.

Accordingly, the invention provides a method for quantitative determination of the number of micro-organisms (or the number of CFUs) in a sample. In many cases, diagnosis and quality control are not based on simple yes/no questions, such as is the micro-organism present or not. In many cases it is necessary to know the amount of the micro-organism present before a decision concerning, e.g. treatment and/or use for production or consumption can be taken. This is generally the case for urine samples, which are never sterile when collected after passage of the urinary tract. Similar questions need to be answered when food, milk and water samples are analysed. Food samples are also rarely completely sterile, and before a decision can be taken about the fate of a batch of food, milk, or water, the microbial count often needs to be known.

The inventors of the present invention have now surprisingly discovered that reproducible and reliable estimates of a microbial count can be determined using a molecular hybridisation assay between a target nucleic acid sequence and a labelled probe. Proper calibration and quantitative recording of the signal produced by the label can estimate a microbial count. One important feature of the invention is the use of LNA monomers in the probes. LNA molecules constitute a class of nucleotide analogues which bind better to both RNA and DNA than do RNA and DNA nucleotides. Therefore more specific binding can be obtained and more stringent washing conditions can be employed whereby the amount of background noise is reduced significantly.

According to an especially preferred embodiment of the invention, the hybridisation assay is performed without the use of any type of genetic amplification such as PCR (polymerase chain reaction) or LCR (ligase chain reaction). The advantages of dispensing with the use of amplification are several. First and foremost one step less is required for the assay, thereby making the assay more simple. Furthermore, amplification reactions, however carefully they are carried out, contribute to the variation. Since in PCR the amplification is exponential, one tiny error in the beginning of the reaction may lead to great differences after a number of rounds of amplification.

A more preferred method of amplification includes signal amplification. Signal amplification may include use of enzymes to produce a detectable product. Alternatively, signal amplification comprises the use of brached DNA detection probes (bDNA). bDNA probes allow for binding of many label probes to each microbial target nucleic acid sequence through hybridisation assays. The result is that a high number of labels are bound to any one target nucleic acid sequence and the amount of signal generated due to the presence of one target nucleic acid is increased. This type of signal amplification is described in U.S. Pat. No. 5,635,352 and U.S. Pat. No. 5,124,246 both of which are assigned to Chiron. Amplification using the techniques described in these references is easier to perform and results in more precise amplification than genetic amplification.

According to a further aspect the invention relates to a kit for determination of a bacterial count in a sample comprising
i. at least one sample compartment,
ii. a lysing solution,
iii. at least one nucleotide probe comprising at least one LNA capable of selectively hybridising to microbial nucleic acid sequences, the at least one probe comprising at least one directly or indirectly quantifiable label.

The kit can be used for performing the method according to the invention. Preferably, the kit comprises standards of known microbial count such as standards of known CFU or with known amount of microbial DNA or with known number of micro-organisms in order to properly calibrate the kit.

Locked nucleic acids represent a class of conformationally restricted nucleotide analogues described in WO 99/14226 (Exiqon), which hybridise stronger to both DNA and RNA than naturally occurring nucleotides. The invention can be carried out with any of the LNAs falling under any of the formulas disclosed in WO 99/14226. The most preferred LNA monomers are those that are commercially available, which have a methyl linker connecting the 2′O position to the 4′C position, such as the one disclosed in FIG. 1.

According to a further aspect, the invention relates to a method for diagnosing an infectious condition in an animal comprising estimating a microbial count in a known volume of a sample using the method according to the invention, and determining whether the count is above or below a certain threshold.

By using the inventive method for estimating a microbial count, results can be obtained more rapidly and reliably than using conventional techniques such as growing on nutrient medium or counting in a microscope. Thus the decision whether to initiate a given treatment and/or to use for consumption and/or to use for production or not can be based on knowledge at an earlier point than hitherto possible.

According to a further aspect the invention relates to a method for quality control of food comprising estimating a microbial count in a known volume of a food sample using the method according to the invention and classifying the sample according to a predetermined standard. One primary advantage of this aspect of the invention is that rapid results can be obtained.

According to a further aspect, the invention relates to a method for quality control of water comprising estimating a microbial count in a known volume of a water sample using the method according to the invention and classifying the sample according to a pre-determined standard.

### FIGURES

FIG. 1. An example of a locked nucleic acid. The figure also shows the basic structure of RNA and DNA for comparison.

FIGS. 2a and 2b are examples of improved nucleic acid hybridisation assays in which capture extender molecules are required to bind a target to the solid support. The figures show different ways in which two capture extenders bind to a single capture probe. CP—capture probe; CE1, CE2, CE3, CE4—capture extenders, LE—label extender, TARGET—microbial target nucleic acid sequence.

FIG. 3 is an example of improved nucleic acid hybridisation assay using label extender molecules that form a cruciform structure. CE—capture extender; LE1, LE2—label extenders; TARGET—microbial target nucleic acid sequence.

FIG. 4 is an example of improved nucleic acid hybridization assay using multiple amplification multimers and bridging label probes. CE—capture extender; LE1, LE2—label extenders; LP—label probe; AMP1, AMP2—amplification multimer.

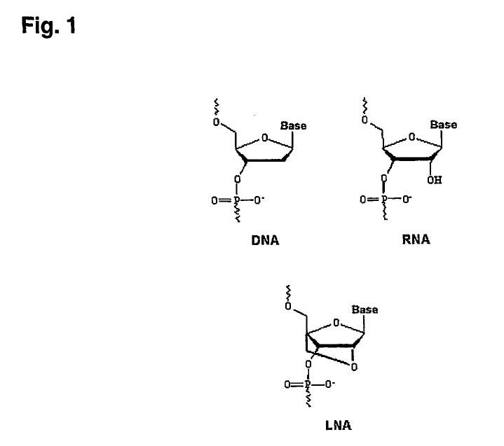

### DEFINITIONS

Micro-organism—fungi, protozoa, algae, bacteria, Archaebacteria, Eubacteria, viruses.

Locked nucleic acid (LNA)—the term refers to the conformationally restricted nucleotide analogues described in WO 99/14226 (Exiqon), in particular those described with reference to formula number I, II, Ia, IIa in the quoted international publication.

Amplifier—refers to a linear or branched polymer of a repeating single-stranded oligonucleotide segment which can hybridise to the same label probe. The repeating single-stranded oligonucleotide segment can be the same or vary slightly from segment to segment as long as they can bind the same label probe under the same preferably high stringency conditions. (U.S. Pat. No. 5,175,270). The amplifier may also consist of several units and can then be referred to as an amplifier multimer.

Capture extender—binds to a target polynucleotide and to capture probes. Thus, capture extender molecules are single-stranded polynucleotide chains having a first polynucleotide sequence region containing a nucleic acid sequence C-1 which is complementary to a sequence in the target polynucleotide and a second region having a capture probe recognition sequence C-2 being complementary to a sequence in the capture probe.

Label extender—contain regions of complementarity vis-a-vis the target polynucleotide and to the amplifier multimer.

### DETAILED DESCRIPTION

The microbial “target nucleic acid” means the microbial derived nucleotide sequence of deoxyribonucleic acid (DNA), ribonucleic acid (RNA) (including messenger ribonucleic acid (mRNA), ribosomal ribonucleic acid (rRNA), transfer RNA, (tRNA), small nuclear (snRNA), telomerase associated RNA, ribozymes etc.) the quantity of which is to be detected in the hybridisation assay. The nucleic acid sample of interest will be one which is suspected of containing a particular target nucleic acid from a particular species of micro-organism, such as a particular gene, gene segment or RNA. Importantly, the invention may assist in the diagnosis of various infectious diseases by assaying for the quantity of target sequences from micro-organisms known to be infectious, the quantity of target sequences being indicative of the number of micro-organisms in the sample. The target nucleic acid may be provided in a complex biological mixture of nucleic acid (RNA, DNA and/or rRNA) and non-nucleic acid. The target nucleic acids of primary preference are RNA molecules and, in particular rRNAs such as the 16S 18S or 23S rRNA. If target nucleic acids of choice are double stranded or otherwise have significant secondary and tertiary structure, they may need to be heated prior to hybridisation. In this case, heating may occur prior to or after the introduction of the nucleic acids into the hybridisation medium containing the capturing probe. It may also be desirable in some cases to extract the nucleic acids from the samples prior to the hybridisation assay to reduce background interference by any methods known in the art.

Samples

The methods are primarily applicable to liquid samples such as urine samples, water samples, liquid food samples such as water, milk or liquid extracts of food samples such as minced meat, environmental samples. However, the method can also be used in connection with solid or semi-solid samples when combined with a step for disruption of the samples, such as sonication or homogenisation. Such solid or semi-solid samples include but are not limited to tissue cultures of animal cells, animal cells (e.g., blood, serum, plasma, reticulocytes, lymphocytes, bone marrow tissue, cerebrospinal fluid, lymph fluid) or any type of tissue biopsy, plant cells and the like. The assay procedures of the present invention are useful, for instance, for determining the quantity of non-pathogenic or pathogenic micro-organisms of interest. By detecting the amount of specific hybridisation between nucleotide probes of a known source and nucleic acids resident in the biological sample, the quantity of the micro-organisms may be established.

Lysis Buffer

Methods for the efficient lysis of nucleotides under conditions which allow hybridisation to be carried out in the lysis solution are disclosed in WO 00/56920 (Exiqon). Solutions containing high concentrations of guanidine, guanine thiocyanate or certain other chaotropic agents and detergents are capable of effectively lysing prokaryotic and eukaryotic cells while simultaneously allowing specific hybridisation of LNA probes to the released endogenous nucleic acid. The solutions need not contain any other component other than common buffers and detergents to promote lysis and solubilisation of cells and nucleic acid hybridisation.

If extraction procedures are employed prior to hybridisation, organic solvents such as phenol and chloroform may be used in techniques employed to isolate nucleic acid. Traditionally, organic solvents, such as phenol or a phenol-chloroform combination are used to extract nucleic acid, using a phase separation (Ausubel et. al in Current Protocols in Molecular Biology, pub. John Wiley & Sons (1998)). These methods may be used effectively with a lysis solution as described herein; however, tedious extraction methods are often not necessary, thus improving the performance of high throughput assays. Preferably, the lysis buffer/hybridisation medium will contain standard buffers and detergents to promote lysis of cells while still allowing effective hybridisation of LNA probes. A buffer such as sodium citrate, Tris-HCl, PIPES or HEPES, preferably Tris-HCl at a concentration of about 0.05 to 0.1 M can be used, more preferably from 10 mM to 1 M, such as from 10 to 100 mM, for example from 40 to 50 mM. The hybridisation medium will preferably also contain about 0.05 to 0.5% (w/v) of an ionic or non-ionic detergent, such as sodium dodecylsulphate (SDS) or Sarkosyl (Sigma Chemical Co., St. Louis, Mo.) and between 1 and 10 mM EDTA. Other additives may also be included, such as volume exclusion agents which include a variety of polar water-soluble or swellable agents, such as anionic polyacrylate or polymethacrylate, and charged saccharidic polymers, such as dextran sulphate and the like. Specificity or the stringency of hybridisation may be controlled, for instance, by varying the concentration and type of chaotropic agent and the NaCl concentration which is typically between 0 and 1 M NaCl, such as 0.1, 0.2, 0.3, 0.4, 0.5, 0.6, 0.7, 0.8, 0.9, or 1.0.

Chaotropic agents which disturb the secondary and tertiary structure of proteins, for example, guanidine salts such as guanidine hydrochloride (GnHCl) and thiocyanate (GnSCN), or urea, lithium chloride and other thiocyanates may be used in combination with detergents and reducing agents such as beta-mercaptoethanol or DTT to dissociate natural occurring nucleic acids and inhibit nucleases. The use of chaotropic agents in the extraction and hybridisation of nucleic acids is described in EP Publication No. 0 127 327, which is incorporated by reference herein.

Preferably the concentration of guanidine hydrochloride lies in the range of 0.1 to 8 M, more preferably from 0.5 to 5 M, more preferably from 2 to 4 M.

LNA Based Probes

A nucleotide probe comprising at least one LNA monomer capable of selectively hybridising to a microbial target nucleic acid sequence is introduced in the hybridisation process. The term “a nucleotide probe comprising at least one LNA monomer capable of selectively hybridising to a microbial target nucleic acid sequence” refers to a polynucleotide or oligonucleotide containing at least one LNA monomer and a variable number of naturally occurring nucleotides or their analogues, such as 7-deazaguanosine or inosine, sufficiently complementary to hybridise with the target nucleic acid such that stable and specific binding occurs between the target and the complementary nucleic acid under the hybridisation conditions.

Exemplary low stringency washing conditions include hybridisation at 42° C. in a solution of 2×SSC, 0.5% (w/v) SDS for 30 minutes and repeating. Exemplary moderate stringency conditions include a wash in 2×SSC, 0.5% (w/v) SDS at 50° C. for 30 minutes and repeating. Exemplary high stringency conditions include a wash in 2×SSC, 0.5% (w/v) SDS, at 65° C. for 30 minutes and repeating. Exemplary very high stringency conditions include a wash in 2×SSC, 0.5% (w/v) SDS, at 70° C. for 30 minutes and repeating. The preferred probes according to the invention are those that hybridise selectively to a target nucleic acid sequence under conditions of high stringency, more preferably under conditions of very high stringency.

Selective hybridisation means that under conditions of high stringency the probes to be used according to the present invention do not hybridise to nucleic acid sequences being at least 70% identical to the microbial target nucleic acid sequences, more preferably at least 75% identical, more preferably at least 80% identical, such as at least 85% identical, for example at least 90% identical, such as at least 95% identical. The use of LNA-based probes allows distinction between sequences having a very high degree of sequence identity.

Therefore, the LNA sequence need not reflect the exact sequence of the target nucleic acid. For example, a non-complementary nucleotide fragment may be attached to a complementary nucleotide fragment or alternatively, non-complementary bases or longer sequences can be interspersed into the complementary nucleic acid, provided that the complementary nucleic acid sequence has sufficient complementarity with the sequence of the target nucleic acid to hybridise therewith, forming a hybridisation complex and further is capable of immobilizing the target nucleic acid to a solid support as will be described in further detail below. A capturing probe to bind the released nucleic acids can be linked to a group (e.g. biotin, fluorescein, magnetic micro-particle etc.). Alternatively, the capturing probe can be permanently bound to a solid phase or particle in advance e.g. by anthraquinone photochemistry (WO 96/31557) or psoralen photochemistry (EP 0 319 957 (GLUETECH APS)).

As for DNA or RNA the degree of complementarity required for formation of a stable hybridisation complex (duplex) which includes LNA varies with the stringency of the hybridisation medium and/or wash medium. The complementary nucleic acid may be present in a pre-prepared hybridisation medium or introduced at some later point prior to hybridisation.

The hybridisation medium is combined with the sample to facilitate lysis of the cells and nucleic acid pairing. Preferably, the volume of sample to the volume of the hybridisation medium will be about 1:10.

It is intended and an advantage of the hybridisation methods of the present invention that they be carried out in one step. However, minor mechanical or other treatments may be considered under certain circumstances. For example, it may be desirable to clarify the lysate before hybridisation such as by slow speed centrifugation or filtration or to extract the nucleic acids before hybridisation as described above.

The hybridisation assay of the present invention can be performed by any method known to those skilled in the art or analogous to immunoassay methodology given in the guidelines presented herein. Preferred methods of assay are the sandwich assays and variations thereof and the competition or displacement assay. Hybridization techniques are generally described in “Nucleic Acid Hybridization, A Practical Approach,” Ed. Hames, B. D. and Higgins, S. J., IRL Press, 1985; Gall and Pardue (1969), Proc. Natl. Acad. Sci., U.S.A., 63: 378-383; and John, Burnsteil and Jones (1969) Nature, 223: 582-587.

In this invention the capturing LNA-probe is typically attached to a solid surface e.g. the surface of a microtiter tray well or a microbead. Therefore a convenient and very efficient washing procedure can be performed thus opening the possibility for various enzymatically based reactions that may add to the performance of the invention.

The hybridisation medium may be pre-prepared, either commercially or in the laboratory to contain all the necessary components for hybridisation. For instance, in a sandwich assay the medium could comprise a chaotropic agent (e.g. guanidine thiocyanate), desired buffers and detergents, a capturing LNA-probe bound to a solid support such as a microbead, and a label probe which is also based on LNA monomers. This medium then only needs to be combined with the sample containing the target nucleic acid at the time the assay is to be performed. Once hybridisation occurs the hybridisation complex attached to the solid support may be washed and the amount of hybridisation determined.

Sandwich Assay

Sandwich assays are commercially useful hybridisation assays for detecting or isolating nucleic acid sequences. Such assays utilise a capture probe covalently immobilised to a solid support and label probe in solution. The sample provides the target nucleic acid. The capture probe and label probe hybridise with the target nucleic acid to form a “sandwich” hybridisation complex. To be effective, the label probe is designed so that it cannot hybridise with the capture probe, but will hybridise with the target nucleic acid in a different position than the capturing probe.

Virtually any solid surface can be used as a support for hybridisation assays, including metals and plastics. Three types of solid surfaces are generally available, namely:
a) Membranes, polystyrene beads, nylon, Teflon, polystyrene/latex beads, latex beads or any solid support possessing an activated carboxylate, sulfonate, phosphate or similar activatable group are suitable for use as solid surface substratum to which nucleic acids or oligonucleotides can be immobilised.
b) Porous membranes possessing pre-activated surfaces which may be obtained commercially (e.g., Pall Immunodyne Immunoaffinity Membrane, Pall BioSupport Division, East Hills, N.Y., or Immobilon Affinity membranes from Millipore, Bedford, Mass.) and which may be used to immobilise capturing oligonucleotides. Micro beads, including magnetic beads, of polystyrene, teflon, nylon, silica or latex may also be used.
c) Plates such as microtiter plates or multiwell dishes, chips, tubes, dipsticks and the like. These are often made of polystyrene. Chips may also be made from glass, and tubes may be made from polyethylene.

However, use of the generally available surfaces mentioned in a) and b) often creates background problems, especially when complex mixtures of nucleic acids and various other dissolved bio-molecules are analysed by hybridisation. A significant decrease in the background has been obtained when the catching-probe is covalently attached to solid surfaces by the anthraquinone (AQ) based photo-coupling method described in the art (see WO 96/31557). This method allows the covalent attachment of the capture probe to the surface of most polymer materials—including various relatively thermostable polymers such as polycarbonate and polyethylene as well as treated glass surfaces.

Sequences for Probes

Sequences suitable for the capture probe or the label probe for use in hybridisation assays can be obtained from the entire sequence or portions thereof of an organism's genome, from messenger RNA, or from cDNA obtained by reverse transcription of messenger RNA. Methods for obtaining the nucleotide sequence from such obtained sequences are well known in the art (see Ausubel et. al in Current Protocols in Molecular Biology, pub. John Wiley & Sons (1998), and Sambrook et al. in Molecular Cloning, A Laboratory Manual, Cold Spring Habor Laboratory Press, 1989). Furthermore, a number of both public and commercial sequence databases are accessible and can be approached to obtain the relevant sequences.

Once the appropriate sequences are determined, LNA probes are preferably chemically synthesised using commercial available methods and equipment as described in the art (Tetrahedron, 1998, 54, 3607-30). For example, the solid phase phosphoramidite method can be used to produce short LNA probes. (Caruthers et al., Cold Spring Harbor Symp. Quant. Biol., 47: 411-418 (1982), and Adams et al., J. Am. Chem. Soc., 105: 661 (1983). When synthesising a probe for a specific target, the choice of nucleotide sequence will determine the specificity of the test. For example, by comparing DNA sequences from several virus isolates, one can select a sequence for virus detection that is either type specific or genus specific. Comparisons of DNA regions and sequences can be achieved using commercially available computer programmes.

Preferred probes according to the invention are such probes that hybridise selectively to the microbial target nucleic acid sequence of interest but not to other nucleic acid sequences present in the sample. Selective hybridisation means that under conditions of high stringency the probes to be used according to the present invention do not hybridise to nucleic acid sequences being at least 70% identical to the microbial target nucleic acid sequences, more preferably at least 75% identical, more preferably at least 80% identical, such as at least 85% identical, for example at least 90% identical, such as at least 95% identical. The use of LNA-based probes allows distinction between sequences having a very high degree of sequence identity.

rRNA Target Sequences

The most preferred target nucleic acid sequences comprise sequences forming part of rRNA. rRNA sequences are very abundant in both prokaryotic and eukaryotic cells so that the amount of target sequence-probe duplex formed is relatively high compared to mRNA and DNA. Compared to mRNA, there are several advantages of using rRNA. First and foremost, the amount of any one species of mRNA varies from cell to cell and over time for any single cell. Furthermore, rRNA molecules are chemically more stable than e.g. mRNA both in the cells and in solution after extraction. Compared to DNA, rRNA first and foremost has the advantage of being more abundant, whereby genetic amplification can be avoided and signal amplification is not required to the same extent as when DNA is used.

Finally, rRNAs contain sequences that are highly conserved across species allowing the design of probes that can be used to quantify the number of micro-organisms belonging to a group. On the other hand rRNAs also contain sequences that vary from species to species, so that species specific probes can be designed.

Determination of Amount of Hybrid

The determination of the amount of hybridisation may be carried out by any of the methods well-known in the art. Typically, labelled signal nucleic acids are used to detect hybridisation. Complementary nucleic acids or signal nucleic acids may be labelled by any one of several methods typically used to detect the presence of hybridised polynucleotides. The most common method of detection is the use of ligands which bind to labelled antibodies, fluorophores or chemiluminescent agents. By far the most preferred labelling methods comprise the use of fluorescent or chemiluminescent probes due to the high sensitivity and low background of such methods. The amount of hybridisation is detected by detecting the amount of signal produced as a result of the hybridisation. Thus, the amount of signal produced must be proportional to the number of probe/target duplexes formed. The amount of signal produced can be detected using conventional techniques and apparatus, which detects e.g. the amount of radiation emitted at a certain wavelength. Conventional apparatus includes Elisa readers, spectrophotometers, absorbance reader, photomultiplier, Charge Coupled Device (CCD), combined with a source of illumination, such as a bulb, light emitting diode (LED), laser and optional filters.

However, probes may also be labelled with ³H, ¹²⁵J, ³⁵S, ¹⁴C, ³³P, or ³²p and subsequently detected by autoradiography. This method is not the most preferred, since it is relatively time-consuming, and due to the inherent dangers associated with the use of radioactive isotops.

Other labels include antibodies which can serve as specific binding pair members for a labelled ligand. The choice of label depends on sensitivity required, ease of conjugation with the probe, stability requirements, and available instrumentation.

LNA-probes are typically labelled during synthesis. Non-radioactive probes are often labelled by indirect means. Generally, a ligand molecule is covalently bound to the probe. The ligand then binds to an anti-ligand molecule which is either inherently detectable or covalently bound to a signal system, such as a detectable enzyme, a fluorescent compound, or a chemiluminescent compound. Ligands and antiligands may be varied widely. Where a ligand has a natural anti-ligand, for example, biotin, thyroxine, and cortisol, it can be used in conjunction with the labelled, naturally occurring anti-ligands. Alternatively, any haptenic or antigenic compound can be used in combination with an antibody.

As is the case of DNA, LNA-probes can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, AMPPD ([3-(2′-spiroamantane)-4-methoxy-4-(3′-phosphoryloxy)-phenyl-1,2-dioxetane]) and 2,3-dihydrophthalazinediones, e.g., luminol.

Signal Amplification

Preferably, the invention comprises the use of some sort of signal amplification. Especially in the case where no genetic amplification is used, is the use of signal amplification advantageous.

Signal amplification is based on techniques known in the art to increase the number of signal molecules linked to any one microbial target nucleic acid sequence. One common way to amplify the amount of signal is to link an enzyme to the target sequence. Through the addition of substrate for the enzyme to the mixture the enzyme can produce a product, which may be detected due to fluorescence or chemiluminescence, absorbance or transmittance. The amount of signal can be increased simply by letting the enzymatic reaction run for a longer period of time.

The amount of signal produced can be further increased by using enzymes conjugated to e.g. two biotin molecules. Thus in a first hybridisation step, a probe conjugated to streptavidin can be linked to the target sequence. In a second amplification step enzymes are added, which are linked covalently to at least two biotin molecules. Each streptavidin may link two, three, or four biotin molecules. Through successive steps of linking streptavidin and enzymes linked to at least two biotin molecules the number of enzymes linked to one target sequence can be increased and the amount of signal which can be ascribed to one target sequence can be increased.

However a more preferred and more precise method comprises the linking several to many chemiluminescent or fluorescent moieties to the microbial target nucleic acid sequence. These methods are more preferred since the ratio of target nucleic acid to the amount of signal produced can be controlled more precisely. One example of this type of signal amplification involves the use of nucleotide amplifiers, label probes, label extenders and optionally capture extenders such as described in U.S. Pat. Nos. 5,635,352 and 5,124,246 (Chiron), which are incorporated by reference in their entirety. Through the use of such techniques, the number of label probes which are linked indirectly to one target nucleic acid can be increased. These methods are extremely precise since the number of signal molecules linked to any one target nucleic acid can be controlled very precisely and the number can furthermore be adjusted to fit the need. Furthermore, due to the high binding affinity between the label probes and the amplifiers, very stringent wash conditions can be employed and the background noise be lowered similarly.

In one embodiment of the invention, an assay is provided in which two or more distinct “capture extender” molecules are used, each of which must bind to the target nucleic acid sequence in order for the assay to result in a detectable signal. As noted above, capture extender molecules are bridging probes which bind to the analyte as well as to support bound “capture probes.” In one embodiment, at least two capture extender molecules must bind to a single support-bound capture probe in order for the assay to result in a detectable signal.

In a further, related embodiment of the invention, an assay is provided in which the melt temperature Tₘ₁ of the multicomponent complex formed between the analyte and support-bound capture probes, mediated by two or more distinct capture extender molecules, is significantly higher than the melt temperature Tₘ₂ of each two-component complex formed between a capture probe and an individual capture extender molecule. In this embodiment, the assay is carried out at conditions which favour formation of hybrid complexes in which the microbial target nucleic acid is bound to the capture probes. This technique is premised on the enhanced stability of the multi-component complex relative to the less stable two-component complexes. A preferred method of favoring analyte-bound hybrid complexes includes running one or more steps of the assay at a temperature between Tₘ₁ and Tₘ₂.

In another embodiment of the invention, an assay is provided in which two or more distinct “label extender” molecules are used; as noted before, label extender molecules are bridging probes which bind to the microbial target nucleic acid sequence as well as to label probes, either directly, as in U.S. Pat. No. 4,868,105, or indirectly through amplification multimers, as in U.S. Pat. No. 5,124,246. Multiple label extenders must bind to the microbial target nucleic acid sequence in order for a quantitative signal (indicating the amount of microorganism in the sample) to be generated.

In another related embodiment of the invention, an assay is provided in which the melt temperature Tₘ₁ of the multicomponent complex formed between the microbial target nucleic acid sequence and an amplification multimer or label probe, mediated by two or more distinct label extender molecules, is significantly higher than the melt temperature Tₘ₂ of each two-component complex formed between an amplification multimer or label probe and an individual label extender molecule. In this embodiment, the assay is carried out at conditions which favour formation of hybrid complexes in which the microbial target nucleic acid sequence is bound to the amplification multimers or label probes. This technique is premised on the enhanced stability of the multi-component complex relative to the much less stable two-component complexes. A preferred method of favoring microbial target nucleic acid sequence-amplification multimer hybrid complexes includes running one or more steps of the assay at a temperature between Tₘ₁ and Tₘ₂.

In still another embodiment of the invention, amplification assays are carried out with two distinct amplification multimers which are bridged by one or more label probes. Each label probe contains two regions, each approximately 5 to 40 nucleotides in length, preferably 10 to 20 nucleotides in length, which are complementary to corresponding regions in each amplification multimer. The length of the complementary regions is selected so as to ensure that the melting temperature of the complex formed between the label probe and a single amplification multimer will be lower, preferably at least about 10° C. lower than the melting temperature of the complex formed between the two amplification multimers, mediated by one or more label probes. Thus, as with the assays described above, an individual multimer will not form a stable hybrid with an individual label probe; however, multi-component hybrid complexes formed from at least one label probe and at least two multimers are stable. Since the multicomponent complex is more likely to form when the amplification multimers are placed in proximity through binding to analyte, this technique produces a more target-dependent signal.

In yet another embodiment of the invention, a variation on the aforementioned assay is provided in which two distinct label probes are provided, wherein the two distinct label probes must bind together in order for a signal to be produced. As with the preceding assays, specificity is enhanced as a result of the additional probe sets and the additional hybridization steps which must take place in order for a detectable signal to be generated.

The invention also encompasses variations on the aforementioned assays, in which, for example, oligonucleotide competitors are incorporated into the assay so as to bind to the capture probes (thus reducing the likelihood of nonspecific hybridization on the solid support), and wherein shorter capture probes are used (again, to reduce the likelihood of nonspecific hybridization on the support). Oligonucleotide competitors may also be used to inhibit binding between the label extenders and the amplification multimers, or between the label probes and the amplification multimers.

Further, the invention encompasses methods for compensating for the loss in signal which can result from the various techniques provided herein for reducing background noise. These methods involve the use of preamplifier molecules which serve as intermediate moieties between label extender molecules and amplification multimers, and are structured so as to bind a plurality of amplification multimers. In this way, the number of label probes per label extender can be vastly increased.

The invention additionally encompasses a method for carrying out a hybridisation assay in which each of the aforementioned techniques are combined, i.e., in which two or more distinct label extender molecules are used, two or more distinct capture extender molecules are used, amplification multimers and label probes are structured such that label probes bridge adjacent multimers, and the like.

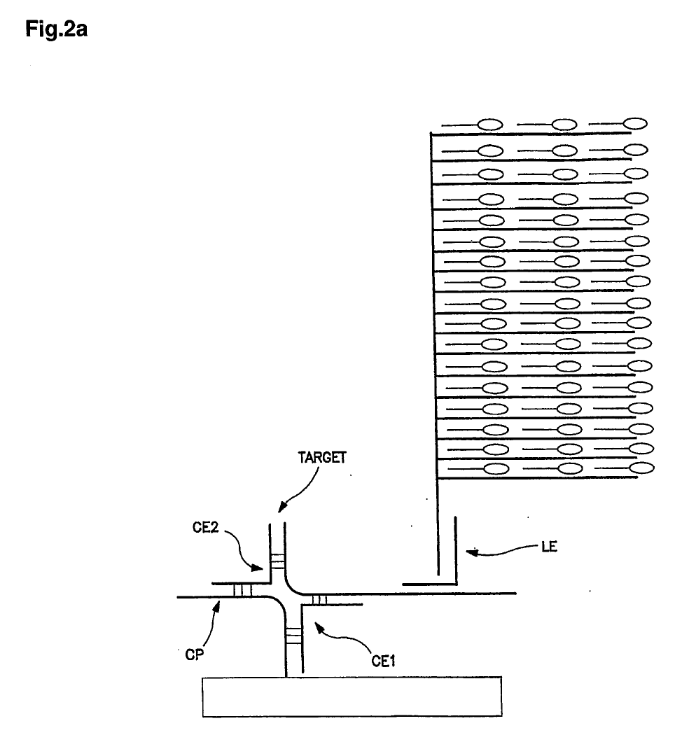

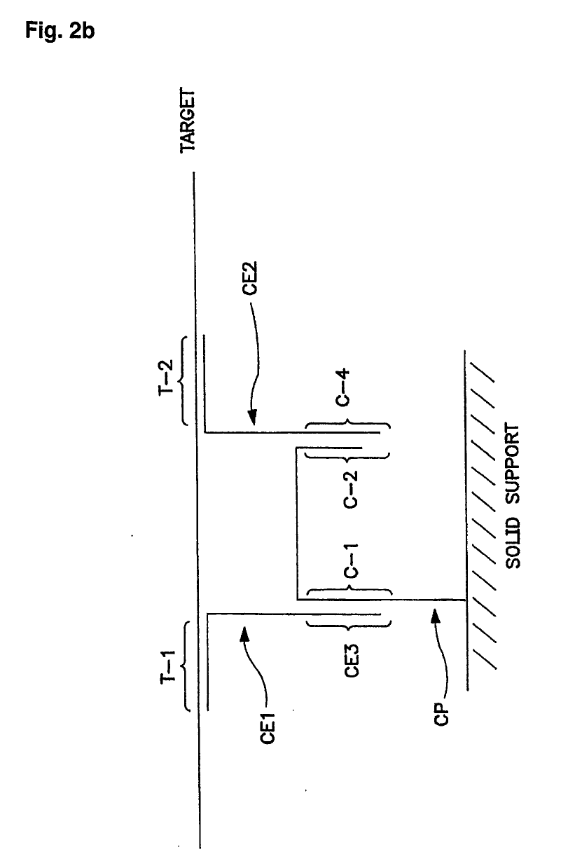

A first embodiment of this assay configuration in which two distinct capture extender molecule are used is illustrated in FIGS. 2a and 2b. In this embodiment, the two distinct capture extenders have distinct first nucleotide sequences complementary to distinct but proximate segments of the target nucleic acid sequence, and also have distinct second nucleotide sequences complementary to distinct segments of a single capture probe. “CE1” and “CE2” represent the two different capture extender molecules, positioned in a cruciform-like structure, such that each extender molecule hybridises to proximate but distinct segments of the target sequence, and to proximate but distinct segments of a single capture probe.

As illustrated in FIG. 2b, the capture probe is structured so as to contain: (1) a first nucleotide sequence C-1 which binds to a nucleotide sequence C-3 in first capture extender CE1; and (2) a different nucleotide sequence C-2 which binds to a nucleotide sequence C-4 in second capture extender CE2. CE1 and CE2 then hybridize to distinct, nonoverlapping segments of the target nucleic acid sequence. Preferably, sequences C-1, C-2, C-3 and C-4 are relatively short, i.e., less than about 30 nucleotides in length, and preferably in the range of about 10 to 15 nucleotides in length. C-1 and C-2 can be directly adjacent, or separated by a spacer region. In addition, it is preferred that the binding of capture probe to capture extender molecules (i.e., C-1:C-3 and C-2:C-4) be relatively weak (Tₘ less than about 55° C.), while the binding of the capture probes to the target nucleic acid sequence through the capture extender molecules be much stronger (Tₘ greater than about 65° C.). This allows the target molecule to bind to the solid support with far greater stability, on the order of 100- to 1000-fold, than the capture extender molecules. This method also enables use of fewer capture probes, which in turn reduces the likelihood of nonspecific hybridisation.

It will be appreciated by those skilled in the art that the cruciform-type configuration shown in FIG. 2a is for purposes of exemplification only, and that alternative assay configurations employing two or more capture extender molecules are also possible. The only requirement is that the assay be structured such that the target binds to the solid support with a melt temperature greater than that of the capture extenders binding to the capture probe. It will also be appreciated by those skilled in the art that the embodiment of FIG. 2b works equally well if C-1 and C-2 are identical capture probe sequences complementary to identical sequences C-3 and C-4 in the two capture extenders: in this instance, the capture probe contains two copies of the repeat sequence C-1.

It will also be appreciated by those skilled in the art that the hybridisation sequences of the capture extenders, label extenders, label probes and amplifier multimers can be based on LNA nucleotide analogues whereby the hybridisation is improved over that of DNA or RNA monomers.

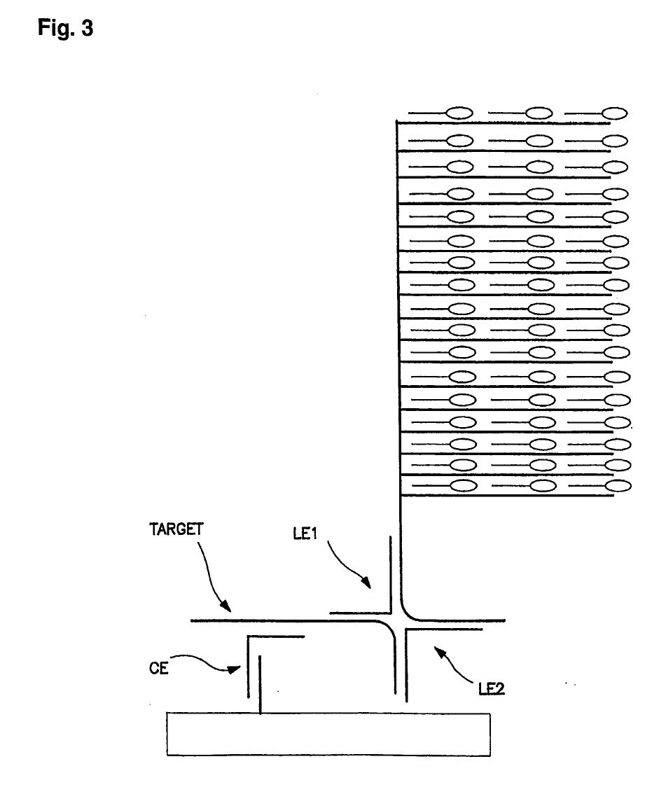

As illustrated in FIG. 3, the amplification multimer is structured so as to contain: (1) a first nucleotide sequence C-1 which binds to a nucleotide sequence C-3 in first label extender LE1; and (2) a different nucleotide sequence C-2 which binds to a nucleotide sequence C-4 in second label extender LE2. LE1 and LE2 then hybridise to distinct, nonoverlapping segments of the microbial target nucleic acid sequence. Preferably, sequences C-1, C-2, C-3 and C-4 are relatively short, i.e., less than about 30 nucleotides in length, and preferably in the range of about 10 to 15 nucleotides in length. C-1 and C-2 can be directly adjacent, or separated by a spacer region. In addition, it is preferred that the binding of amplification multimer to label extender molecules (i.e., C-1:C-3 and C-2:C-4) be relatively weak (Tₘ less than about 45°), while the binding of the amplification multimer to the target through the label extender molecules be much stronger (Tₘ greater than about 65°). This allows the target molecule to bind to the amplification multimer with far greater stability, on the order of 100- to 1000-fold, than the label extender molecules. Again, as with the preceding method in which at least two capture extender molecules are used, assay specificity is increased by virtue of the additional hybridisation steps which are necessary to give rise to a target-dependent signal.

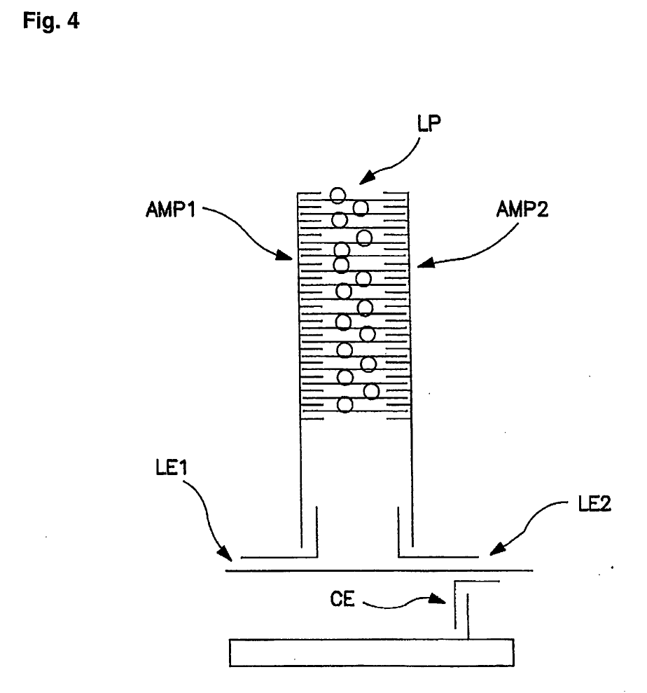

In another embodiment of the invention, the phenomenon of target-independent signal generation is addressed by bridging adjacent amplifier molecules in such a way as to reduce virtually all of the principal sources of assay background, including nonspecific hybridisation of label extender molecules to capture probes and capture extender molecules, nonspecific hybridisation of amplification multimers to capture probes and capture extender molecules and amplifier nonspecific binding. In this embodiment, two distinct amplifier multimers are used, designated AMP1 and AMP2 in FIG. 4, as well as two distinct label extender molecules, designated LE1 and LE2. Neither AMP1 nor AMP2 will retain label unless they are within bridging distance of each other, so that there is a much higher likelihood that the amplifiers are actually bound to the target molecule before labelling occurs. This is accomplished by providing label probes which contain: (1) a first nucleic acid sequence L-1 which contains a nucleic acid sequence complementary to a region in the repeating oligonucleotide subunits of AMP1; (2) a second nucleic acid sequence L-2 which contains a nucleic acid sequence complementary to a region in the repeating oligonucleotide subunits of AMP2; and (3) a detectable label therebetween. L-1, L-2, and the corresponding complementary sequences in the amplifier probes are selected such that the melting temperature of the complex formed from both amplifier probes and the label probes is preferably at least about 10° C. higher than the melting temperature of the complex formed between the label probe and a single amplifier multimer. It will also be appreciated that such a configuration gives rise to the advantages discussed above with respect to the use of multiple probes and consequent additional hybridisation steps required to produce a detectable signal.

The amount of labelled probe which is present in the hybridisation medium or extraction solution may vary widely. Generally, substantial excesses of probe over the stoichiometric amount of the target nucleic acid will be employed to enhance the rate of binding of the probe to the target DNA. Treatment with ultrasound by immersion of the reaction vessel into commercially available sonication baths can often accelerate the hybridisation rates.

Wash Solution and Conditions

After hybridisation at a temperature and time period appropriate for the particular hybridisation solution used, the support to which the capturing LNA-probe: target nucleic acid hybridisation complex is attached is introduced into a wash solution typically containing similar reagents (e.g., sodium chloride, buffers, organic solvents and detergent), as provided in the hybridisation solution. These reagents may be at similar concentrations as the hybridisation medium, but often they are at lower concentrations when more stringent washing conditions are desired. The time period for which the support is maintained in the wash solutions may vary from minutes to several hours or more.

Either the hybridisation or the wash medium can be stringent according to the conditions mentioned above or in the appended examples. After appropriate stringent washing, the correct hybridisation complex may now be detected in accordance with the nature of the label. Due to use of probes comprising at least one LNA nucleotide analogue it is actually possible to wash in pure water, i.e. under conditions of extremely high stringency. Therefore, the amount of unspecific binding is reduced to an absolute minimum and background noise is reduced accordingly.

Label-Probe Conjugation

The probe may be conjugated directly with the label. For example, where the label is radioactive, the probe with associated hybridisation complex substrate is exposed to X-ray film. Where the label is fluorescent, the sample is detected by first irradiating it with light of a particular wavelength. The sample absorbs this light and then emits light of a different wavelength which is picked up by a detector (Physical Biochemistry, Freifelder, D., W. H. Freeman & Co. (1982), pp. 537-542). Where the label is an enzyme, the sample is detected by incubation on an appropriate substrate for the enzyme. The signal generated may be a coloured precipitate, a coloured or fluorescent soluble material, or photons generated by bioluminescence or chemiluminescence. The preferred label for probe assays comprises a fluorescent or chemiluminescent label due to the high intensity of such labels, the absence of background signal and the possibility to quantify the result, which is importanct in the assays according to the present invention.

Detection of a hybridisation complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The label may also allow indirect detection of the hybridisation complex. For example, where the label is a hapten or antigen, the sample can be detected by using antibodies. In these systems, a signal is generated by attaching fluorescent or enzyme molecules to the antibodies or in some cases, by attachment to a radioactive label. (Tijssen, P., “Practice and Theory of Enzyme Immunoassays, “Laboratory Techniques in Biochemistry and Molecular Biology, Burdon, R. H., van Knippenberg, P. H., Eds., Elsevier (1985), pp. 9-20.)

Labels

In the present context, the term “label” thus means a group which is detectable either by itself or as a part of an detection series. Examples of functional parts of reporter groups are biotin, digoxigenin, fluorescent groups (groups which are able to absorb electromagnetic radiation, e.g. light or X-rays, of a certain wavelength, and which subsequently re-emits the energy absorbed as radiation of longer wavelength; illustrative examples are dansyl (5-dimethylamino)-1-naphthalenesulfonyl), DOXYL (N-oxyl-4,4-dimethyloxazoli-dine), PROXYL (N-oxyl-2,2,5,5-tetramethylpyrrolidine), TEMPO (N-oxyl-2,2,6,6-tetra-methylpiperidine), dinitrophenyl, acridines, coumarins, Cy3 and Cy5 (trademarks for Biological Detection Systems, Inc.), erytrosine, coumaric acid, umbelliferone, Texas Red, rhodamine, tetramethyl rhodamine, Rox, 7-nitrobenzo-2-oxa-1-diazole (NBD), pyrene, fluorescein, Europium, Ruthenium, Samarium, and other rare earth metals), chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy), enzymes (such as peroxidases, alkaline phosphatases, galactosidases, and glycose oxidases), antigens, antibodies, haptens (groups which are able to combine with an antibody, but which cannot initiate an immune response by themselves, such as peptides and steroid hormones), carrier systems for cell membrane penetration such as: fatty acid residues, steroid moieties (cholesteryl), vitamin A, vitamin D, vitamin E, folic acid peptides for specific receptors, groups for mediating endocytose, epidermal growth factor (EGF), bradykinin, and platelet derived growth factor (PDGF). Especially interesting examples are biotin, fluorescein, Texas Red, rhodamine, dinitrophenyl, digoxigenin, Ruthenium, Europium, Cy5, Cy3, etc.

RNA-LNA Interaction

The processes for conducting nucleic acid hybridisations wherein the target nucleic acid is RNA comprise heating a nucleic acid solution or sample to an elevated temperature e.g. 70-100° C. as described in the art (U.S. Pat. No. 5,376,529). The nucleic acid solution of the present invention may comprise a chaotropic agent, a target nucleic acid, and a LNA nucleotide probe substantially complementary to the target nucleic acid of interest. The nucleic acid solution will be heated to fully disrupt the protein and nucleic acid interactions to maximise hybridisation between the LNA and its target.

When very high affinity LNA probes are used, hybridisation may take place even at the increased temperature needed to fully disrupt DNA:DNA and DNA:RNA interactions. The solution is then cooled until the complementary nucleic acid has hybridised with the target nucleic acid to form a hybridisation complex.

These methods are advantageous because they allow for minimal handling of the samples and assay reagents. A ready-to-use reagent solution may be provided, for example, which would contain a chaotropic agent, other appropriate components such as buffers or detergents, a capturing LNA-probe bound to a solid support, and a signal or detection LNA (or nucleic acid), both capable of hybridising with a target nucleic acid.

Dynamic Range

The dynamic range of the method is intended to lie within 10 to 10⁷ micro-organisms per mL, more preferably within 10² to 10⁶ micro-organisms per mL. The upper limit of the dynamic range is primarily determined by the number of capture probes used to capture the microbial target nucleic acid. The lower limit is primarily determined by the sensitivity of the hybridisation and detection.

Preferably, the dynamic range of the method is within 10² to 10⁵ micro-organisms per mL of sample. For example the dynamic range of the method lies is within 10 to 10⁶, such as within 10² to 10³, 10³ to 10⁴, 10⁴ to 10⁵, 10⁵ to 10⁶, 10² to 10⁶, 10² to 10⁴, 10³ to 10⁶, 10³ to 10⁵, or within 10⁴ to 10⁶ micro-organisms per mL of sample.

Correlation of the Result

Correlation of the result of the determination of the amount of hybrid to the microbial count can be performed e.g. by comparing the recorded result to the results of a known sample. According to one embodiment, this may be done by establishing a standard curve using samples with known microbial count and converting the result of the detection to a microbial count using the standard curve.

Calibration

In order to obtain reproducible and precise results, the method is calibrated prior to performing the correlation.

Calibration may comprise determination of the amount of hybrid in samples with known microbial counts. Calibration may likewise comprise determination of the amount of hybrid in samples with known amounts of microbial DNA. Finally, calibration may comprise determination of the amount of hybrid in samples with known amounts of colony forming units.

Irrespective of the type of samples used for the calibration, calibration is preferably performed with samples lying within the dynamic range of the method. The calibration standards should also cover the whole of the dynamic range.

Types of Assay

In principle two types of assay can be performed. One type of assay is a species specific assay, wherein the result obtained is a microbial count of one species of micro-organism. In such cases, the probe selectively hybridises to a target nucleotide sequence from one species of micro-organism, and the result is the number of this one species of micro-organism.

Examples of the one species of micro-organism include but are not limited to the group consisting of E. coli, Enterococcus faecalis, Staphylococcus aureus, Staphylococcus saprophyticus, Salmonella spp., Campylobacter spp., Legionella spp., Listeria spp., Klebsiella pneumoniae, K. oxytoca, Proteus spp., Proteus mirabilis, Enterobacter spp,. E. cloacae, Serratia spp., S. marcescens, Citrobacter spp., C. freundii, Morganella morganii, Pseudomonas spp., Pseudomonas aeruginosa, Cryptosporidium parvum, Giardia intestinalis, Plasmodium falciparum, P. vivax, P. ovale, P. malariae, Enterococcus spp., E. faecium.

The other type of assay is an assay for a group of micro-organisms. Here, the probe selectively hybridises to microbial target nucleic acid sequences, which are sufficiently conserved across a group of species of micro-organism and the result is the number of micro-organisms belonging to said group.

Examples of groups of micro-organism include but are not limited to the group consisting of Eubacteriae, Enterobacteriaceae, Listeria spp., Enterobacter spp., Salmonella spp., Campylobacter spp., Enterococcus spp., Plasmodium spp, Staphylococcus spp, S. aureus, Legionella spp, Klebsiella spp, Proteus spp, Serratia spp, Citrobacter spp, Morganella spp, Pseudomonas spp, Cryptosporidium spp, Giardia spp.

Kits

Kits for the determination of a microbial count are also contemplated. Such kits would contain at least one vial containing an extraction solution or a hybridisation medium which comprises a capturing LNA probe bound to a solid support. Detergents, buffer solutions, and additional vials which contain washing solutions, and components to detect target nucleic acids may also be included. Preferably, a strong chaotropic agent is included in the hybridisation medium.

The kits may be designed to fit specific purposes. Thus it is contemplated that one kit is made for estimating microbial counts in urine samples. Such a kit would contain probes to detect the total number of Eubacteria, the number of E. coli, S. aureus, and Enterococcus spp, Enterobacter spp, Klebsiella pneumoniae, Proteus mirabilis, and optionally Candida albicans. Conveniently such a kit also comprises the calibration standards allowing calibration and analysis to be carried out in one step. Such a kit would conveniently be laid out as a microtiter plate, which could be run on standard laboratory equipment both for hybridisation, wash and detection.

The calibration standards could be in the form of pre-determined amount of freeze dried micro-organism or nucleotide preparations from these. Calibration standards may also be quality control standards corresponding bacterial count limits set by authority (e.g. in food, milk, drinking water and waste water analysis).

Another kit may be designed for water analysis. Such a kit could include probes selective for Cryptosporidium parvum, Giardia intestinalis, and Legionella spp. It could be laid out as the other kit described above.

A kit for food analysis could include probes selective for Salmonella spp, Salmonella DT104, Campylobacter spp., E. coli, E. coli 0157, and Listeria spp, L. monocytogenes, as well as calibration standards.

A kit for analysis of milk samples includes probes for detection of the total amount of bacteria, for detection of the amount of Staphyllococcus spp, Streptococcus spp and S. aureus. The kit may also contain probes for detection the amount of E. coli Klebsiella spp., and Pseudomonas spp. Calibration standards may include standards with known amounts of bacteria. Furthermore the kits may contain information to convert the amount of bacteria to an amount of somatic cells since this is the standard usually employed in the art.

A kit for analysis of blood samples suspected of containing malaria could include probes to detect the amount of Plasmodium falciparum, P. vivax, P. ovale, and P. malariae in a known amount of erythrocytes as well as calibration standards.

The physical layout of the kit according to the invention may be a kit wherein the capture probe is bound to a solid surface, such as a well, a chip, a dipstick or a bead, such as a magnetic bead.

Preferably the kit comprises a microtiter plate or a multiwell dish adapted to be used with an Elisa-reader, and the capture probe is bound to the surface of a well, the kit further comprising washing solution, and a hybridisation solution comprising a label probe.

Characteristics of Nucleotide Probes Comprising at Least One LNA

The probes used in the methods and kits according to the present invention contain at least one LNA nucleotide analogue. Preferably, only the part of the probes, which hybridises with the microbial target nucleic acid contain LNA analogue(s). The recognition sequence of the probes (both the capture probe, the label probe, optional capture and label extenders and amplifiers) typically comprise from 4 to 50 nucleotides, more preferably from 7-30 nucleotides, more preferably from 7 to 20, such as from 8 to 15 nucleotide. Of these, from 10 to 100% are LNA nucleotide analogues, and the remaining may be DNA, RNA or any other type of nucleotide or nucleotide analogue. Thus one recognition sequence may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or even 50 LNA nucleotide analogues.

The longer the recognition sequence the lower is the percentage of LNA analogues required in the sequence to obtain the degree of hybridisation required to perform efficient washing. Thus with relatively short recognition sequences, such as below 10 nucleotides, it is contemplated that the percentage of LNA analogues is 80, 90, or 100.

The recognition sequence is preferably at least 70% identical to the microbial target sequence, more preferably at least 80% identical, such as at least 90% identical, for example at least 95% identical, such as at least 98% identical, for example 100% identical.

The probes may furthermore comprise spacer sequences comprising from 5 to 50 nucleotides, which do not hybridise to other sequences in the sample. The capture probes may also contain a linker sequence to link the nucleotide sequence to an anthraquinone or psoralen moiety for linking it to a solid surface. This linker portion may be a linear alkyl moiety with from 2 to 10 carbon atoms or it may be a linker of the general formula ((CH₂)ₙO)ₘ, wherein n is from 1 to 4 and m is from 2 to 10, preferably wherein n is 2 and m is from 3 to 7.

A linear alkyl linker may also be used to link the label probe to the label portion of the probe or to any member of a species specific linkage being covalently linked to said label probe.

The probes used according to the present invention are also intended to cover chimeric probes. The term “chimeric probes” means two or more oligomers with monomers of different origin joined either directly or via a spacer. Illustrative examples of such oligomers which can be combined are peptides, PNA-oligomers, oligomers containing LNAs, and oligonucleotide oligomers.

Generally, the oligomers comprising LNA monomers have surprisingly good hybridisation properties with respect to affinity and specificity. Thus, the oligomers comprise at least one nucleoside analogue which imparts to the oligomer a Tₘ with a complementary DNA oligonucleotide which is at least 2.5° C. higher, preferably at least 3.5° C. higher, in particular at least 4.0° C. higher, especially at least 5.0° C. higher, than that of the corresponding unmodified reference oligonucleotide which does not comprise any nucleoside analogue. In particular, the Tₘ of the oligomer is at least 2.5×N° C. higher, preferably at least 3.5×N° C. higher, in particular at least 4.0×N° C. higher, especially at least 5.0×N° C. higher, where N is the number of nucleoside analogues.

In the case of hybridisation with a complementary RNA oligonucleotide, the at least one nucleoside analogue imparts to the oligomer a Tₘ with the complementary RNA oligonucleotide which is at least 4.0° C. higher, preferably at least 5.0° C. higher, in particular at least 6.0° C. higher, especially at least 7.0° C. higher, than that of the corresponding unmodified reference oligonucleotide which does not comprise any nucleoside analogue. In particular, the Tₘ of the oligomer is at least 4.0×N° C. higher, preferably at least 5.0×N° C. higher, in particular at least 6.0×N ° C. higher, especially at least 7.0×N ° C. higher, where N is the number of nucleoside analogues.

The term “corresponding unmodified reference oligonucleotide” is intended to mean an oligonucleotide solely consisting of naturally occurring nucleotides which represents the same nucleobases in the same absolute order (and the same orientation).

The Tₘ is measured under one of the following conditions:
a) 10 mM Na₂HPO₄, pH 7.0, 100 mM NaCl, 0.1 mM EDTA;
b) 10 mM Na₂HPO₄ pH EDTA;
c) 3 M tetramethylammoniumchloride (TMAC), 10 mM Na₂HPO₄, pH 7.0, 0.1 mM EDTA;
   preferably under conditions a), at equimolar amounts (typically 1.0 M) of the oligomer and the complementary DNA oligonucleotide.

Furthermore, with respect to specificity and affinity, the oligomer, when hybridised with a partially complementary DNA oligonucleotide, or a partially complementary RNA oligonucleotide, having one or more mismatches with said oligomer, should exhibit a reduction in Tₘ, as a result of said mismatches, which is equal to or greater than the reduction which would be observed with the corresponding unmodified reference oligonucleotide which does not comprise any nucleoside analogues. Also, the oligomer should have substantially the same sensitivity of Tₘ to the ionic strength of the hybridisation buffer as that of the corresponding unmodified reference oligonucleotide.

Oligomers defined herein are typically at least 1% modified, such as at least 2% modified, e.g. 3% modified, 4% modified, 5% modified, 6% modified, 7% modified, 8% modified, or 9% modified, at least 10% modified, such as at least 11% modified, e.g. 12% modified, 13% modified, 14% modified, or 15% modified, at least 20% modified, such as at least 30% modified, at least 50% modified, e.g. 70% modified, and in some interesting applications 100% modified.

The oligomers preferably have substantially higher 3′-exonucleolytic stability than the corresponding unmodified reference oligonucleotide.

It should be understood that oligomers (wherein LNAs are incorporated) and LNAs as such include possible salts thereof, of which pharmaceutically acceptable salts are especially relevant. Salts include acid addition salts and basic salts. Examples of acid addition salts are hydrochloride salts, sodium salts, calcium salts, potassium salts, etc. Examples of basic salts are salts where the (remaining) counter ion is selected from alkali metals, such as sodium and potassium, alkaline earth metals, such as calcium, and ammonium ions. Pharmaceutically acceptable salts are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the oligomers and LNAs as well as any intermediates or starting materials therefor may also be present in hydrate form.

Diagnosing

The methods according to the present invention are conveniently used for diagnosing various infectious conditions in animals including human beings. Very often diagnosis is not based merely on the presence or absence of a specific micro-organism but rather on the amount of such a micro-organism present in a specific sample. One well-known example of such a condition includes urine samples. Due to the sampling-techniques, there can be micro-organisms in a sterile urine sample, and more specifically there can be E. coli in urine samples. The presence of micro-organisms including E. coli do not represent any substantial problems in itself. Only when the bacterial count exceeds a certain threshold in combination with the patient presenting relevant clinical symptoms, is an antibiotic treatment initiated. Another well known example is mastitis caused by bacterial infection of the udder. All milk samples contain bacteria but only those with a bacterial count above a given threshold are indicative of mastitis.

It is also contemplated that the method can be used for determining the number of Plasmodium sp. or the number of P. falciparum, P. ovale, P. vivax, or P. malariae in a known amount of erythrocytes. In the treatment of malaria the number of micro-organisms in a blood sample is a very important parameter and more important than the mere detection of presence or absence.

A further example of diagnosis comprises Paratuberculosis in a ruminant, and where the quantitative detection comprises estimation of the number of Mycobacterium paratuberculosis in a known volume of ruminant faeces.

Further examples include, but are not limited to udder infection (mastitis) in a lactating animal, such as a cow, goat or sheep, where the quantitative determination comprises estimation of the number of bacteria in a known volume of a milk sample.

Food Control

A further application of the methods according to the invention comprises quality control of food performed by estimating a microbial count in a known volume of a food sample and classifying the sample according to a predetermined standard. The industrialised world has seen a very high incidence of infections caused by the presence of pathogenic micro-organisms in food samples, such as the presence of Salmonella spp, E coli, or E coli 0157 in meat, in particular in minced meat, Listeria spp in particular L. monocytogenes in cheese, milk, and other dairy products. Quality control performed in the industry, butcheries, restaurants, and shops include sampling of samples and subsequent plating of these samples or extracts thereof on microbiological media. When using the methods according to the present invention, the results of such sampling can be obtained within hours instead of within days.

Other types of samples include beverages such as juice, soft drinks, mineral water, and the like.

Water Quality Control

A further application comprises quality control of water comprising estimating a microbial count in a known volume of a water sample using the method according to the invention and classifying the sample according to a pre-determined standard.

Water samples are examined today using microscopic observation as well as growth on nutrient media to estimate the number of CFU.

The water sample may comprise waste water, bathing water, fountain water, humidifier water, cooling water, man-made water reservoir, drinking water, and tap water.

In particular the tap water may be obtained from or intended for a hospital, a health care facility, a hotel, a ship, a cruise ship, a yacht, an aeroplane, a train, or tourist facilities. When analysing water samples, the microbial count often is the number of Legionella spp, L. pneumoniae, Cryptosporidium parvum, or Giardia intestinalis.

Cell Number Estimation During Fermentation

The methods according to the present invention can also be used for estimation of cell numbers during microbial fermentation, employing the use of bacteria or fungi.

### EXAMPLES

Immobilisation of Capture Probes:

Anthraquinone LNA capture probes are dissolved in 0.2 M NaCl equivalent to a concentration of 0.1 microM. 100 microlitres are added to each well in a microtiterplate (C96 polysorp, Nalge Nunc International, Roskilde, Denmark) and is exposed to “soft” UV-light (approx. 350 nm) in a UV illuminator for 15 minutes. The plates are, thereafter, washed with 300 microlitres 0.4 M NaOH mixed with 0.25% Tween 20 followed by three washes with 300 microlitres of deionized water.

Lysing of Bacterial Cells

1 ml urine centrifuged at 3000×g for 10 minutes. The pellet is dissolved in 25 microlitres of lysosyme solution (50 mM Tris HCl pH 8, 250 mM EDTA pH 8, 1.5 mg/ml lysozyme, 0.1% sucrose) and is incubated on ice for 15 min. 375 microlitres of GnSCN buffer is, thereafter, added (2 M GnSCN, 40 mM NaCitrate (pH=7), 0.5% Sarcosyl).

Hybridisation with Target and Detection Probe:

100 microlitres of prepared target is added to each well (hybridisation buffer: 2 M GnSCN, 40 mM NaCitrate (pH=7), 0.5% Sarcosyl) and is incubated for 30 minutes at 37°. Subsequently, the wells are washed five times with 300 microlitres washing buffer (1×SSC, 0.1% Tween 20 (1×SSC=150 mM NaCl, 150 mM NaCitrat)). Then 100 microlitres 0.12 microM biotinylated detection probe is added (detection buffer; 1×SSC, 0.1% Tween 20), which is incubated for 30 minutes at 37°. Finally, the wells are washed three times with 250 microlitres 1×SSC, 0.1% Tween 20.

Formed hybrids are detected by binding of streptavidin-horseradish peroxidase (1 microgram/mL dissolved in 1×SSC, 0.1% Tween 20) to the biotinylated detection probe. 100 microL streptavidin-horseradish peroxidase solution is added to each well and incubated at 37° C. for 15 minutes. The wells are washed three times with 250 microlitres 1×SSC, 0.1% Tween 20, after which the signal is produced in an OPD assay.

OPD Assay:

100 microlitres of mastermix (6 ml 0.1 M citratebuffer pH=5, two 2 mg ortho-phenylene-diamine (OPD) tablets, 2.5 microlitres 30% H₂O₂) is added to each well and incubated at 22° C. between 1 and 30 minutes, depending on the enzyme activity. The reaction is stopped with 100 microlitres 0.5 M H₂SO₄, and the optical density is measured at 492 nm in an ELISA reader.

Capture Probes:

| | | |
|---|---|---|
| Universal: | 5′ AGGAGGTGATCCAACCGCA3′ | (SEQ ID No 1) |

Detection probes:

| | |
|---|---|
| Enterobacteriaceae: | |
| 5′GGCGCTTACCACTTTGTGATTCATG3′ | (SEQ ID No 2) |
| E. coli - ECA75F: | |
| 5′GGAAGAAGCTTGCTTCTTTGCTGAC3′ | (SEQ ID No 3) |

Species Specific Probes.

The following rRNA specific probes can be biotinylated and used to hybridise specifically to rRNA sequences

| | |
|---|---|
| Staphylococcus aureus | |
| cctttgacaactctagagatagag | (SEQ ID No 4) |
| Salmonella spp. | |
| ttggtaagccgggatggccc | (SEQ ID No 5) |
| Salmonella spp. 16S | |
| tccacagaga tccagagatg gattttcttc | (SEQ ID No 6) |
| ggaac | |
| Enterobacteriaceae: | |
| TGCTCTCGCGAGGTCGCTTCTCTT | (SEQ ID No 7) |

The following probe sets (left primer, right primer, hyb oligo) can be used for amplification of DNA (using the left and right primer) and subsequent detection with the hybridisation oligo, which is biotinylated.

The sequences can also be used for preparing biotinylated probes for direct (without amplification) detection of rRNA. For this embodiment either the left primer or the right primer can be used.

The probes can be used in combination with the universal capture probe disclosed above.

| | | |
|---|---|---|
| S. aureus | | |
| LEFT PRIMER | gaaagccacggctaactacg | (SEQ ID No 8) |
| RIGHT PRIMER | catttcaccgctacacatgg | (SEQ ID No 9) |
| HYB OLIGO | gcaagcgttatccggaatta | (SEQ ID No 10) |

L. monocytogenes

| | | |
|---|---|---|
| LEFT PRIMER | cccttatgacctgggctaca | (SEQ ID No 11) |
| RIGHT PRIMER | cctaccgacttcgggtgtta | (SEQ ID No 12) |
| HYB OLIGO | tgcaactcgcctacatgaag | (SEQ ID No 13) |

Cryptosporidium parvum

| | | |
|---|---|---|
| LEFT PRIMER | atggccgttcttagttggtg | (SEQ ID No 14) |
| RIGHT PRIMER | ccatttccttcgaaacagga | (SEQ ID No 15) |
| HYB OLIGO | ttccgttaacgaacgagacc | (SEQ ID No 16) |
| Giardia | | |
| LEFT PRIMER | gacgggtgaaacaggatgat | (SEQ ID No 17) |
| RIGHT PRIMER | ccgtcaatgccttcaagttt | (SEQ ID No 18) |
| HYB OLIGO | agaaggcgatcagacaccac | (SEQ ID No 19) |

Salmonella spp. 23S

| | | |
|---|---|---|
| LEFT PRIMER | tgcgaaagcaggtcatagtg | (SEQ ID No 20) |
| RIGHT PRIMER | acctacttcagccccaggat | (SEQ ID No 21) |
| HYB OLIGO | aagagttcatatcgacggcg | (SEQ ID No 22) |

Salmonella 16S RNA

| | | |
|---|---|---|
| LEFT PRIMER | cagccacactggaactgaga | (SEQ ID No 23) |
| RIGHT PRIMER | gttagccggtgcttcttctg | (SEQ ID No 24) |
| HYB OLIGO | aggccttcgggttgtaaagt | (SEQ ID No 25) |

Campylobacter jejuni 16S RNA

| | | |
|---|---|---|
| LEFT PRIMER | gtccccagcaaacaggatta | (SEQ ID No 26) |
| RIGHT PRIMER | ccgaaccgttagcaacaaat | (SEQ ID No 27) |
| HYB OLIGO | aacgcattaagtgtaccgcc | (SEQ ID No 28) |

1-110. (canceled)

111. A method for quantitative determination of the number of micro-organisms in a sample, without any amplification of the target nucleic acid sequences comprising
providing a sample suspected of containing at least one species of micro-organism, lysing the micro-organisms in the sample,
contacting the sample with a t least one nucleotide-probe comprising at least one locked nucleic acid (LNA) and being capable of selectively hybridising to a microbial target nucleic acid sequence,
determining the amount of hybrid,
correlating the result to the number of at least one species of micro-organism in the sample to generate a dynamic range within 10 to 10⁴ micro-organisms per ml.

112. The method according to claim 111, wherein said target sequence comprises 16S or 18S or 23S nucleic acid sequence or a sequence coding for 16S or 18S or 23S rRNA.

113. The method according to claim 111, wherein the nucleotide-probe comprises a sequence selected from the group SEQ ID No 1 to 28.

114. The method according to claim 111, wherein the probe selectively hybridises to a target nucleotide sequence from one species of micro-organism, and the result is the number of this one species of micro-organism.

115. The method according to claim 111, wherein the probe selectively hybridises to a target nucleic acid sequence from a group of species of micro-organism and the result is the number of micro-organisms belonging to said group.

116. The method according to claim 111, wherein at least one probe is linked to a label moiety, said probe being a label probe.

117. The method according to claim 116, wherein the label probe comprises a recognition sequence and a linker portion.

118. The method according to claim 117, wherein the label probe further comprises at least one spacer oligonucleotide sequence.

119. The method according to claim 111, further comprising calibration of the method prior to performing the correlation.

120. A kit for quantitative determination of the number of micro-organisms in a sample comprising
at least one sample compartment,
a lysing solution,
at least one nucleotide probe comprising at least one LNA capable of selectively hybridising to microbial nucleic acid sequences, the at least one probe comprising at least one directly or indirectly quantifiable label, and
standards of known microbial count,
without any amplification of the target nucleic acid sequences.

121. The kit according to claim 120, wherein the standards comprise freeze dried samples of the micro-organisms.

122. The kit according to claim 120 comprising several nucleotide probes for the simultaneous estimation of several microbial counts, the several nucleotide probes being spatially separated.

123. A method for diagnosing an infectious condition in an animal comprising estimating a microbial count using the method according to claim 111, and determining whether the count is above or below a certain threshold.

124. The method according to claim 123, wherein the infectious condition comprises a urinary tract infection, and the method comprises estimation of the number of bacteria in a known volume of urine sample, such as the number of E. coli.

125. A method for quality control of food comprising estimating a microbial count in a known amount of food sample using the method according to claim 111 and classifying the sample according to a predetermined standard.

126. A method for quality control of water comprising estimating a microbial count in a known volume of water sample using the method according to claim 111 and classifying the sample according to a pre-determined standard.


SEQUENCE LISTING (28 sequences)

SEQ ID NO: 1 - DNA, 19 bp, Artificial Sequence
  Universial capture probe
aggaggtgatccaaccgca

SEQ ID NO: 2 - DNA, 25 bp, Enterobacteriaceae
ggcgcttaccactttgtgattcatg

SEQ ID NO: 3 - DNA, 25 bp, E coli
ggaagaagcttgcttctttgctgac

SEQ ID NO: 4 - DNA, 24 bp, Staphylococcus aureus
cctttgacaactctagagatagag

SEQ ID NO: 5 - DNA, 20 bp, Salmonella spp
ttggtaagccgggatggccc

SEQ ID NO: 6 - DNA, 35 bp, Salmonella spp
tccacagagatccagagatggattttcttcggaac

SEQ ID NO: 7 - DNA, 24 bp, Enterobacteriaceae
tgctctcgcgaggtcgcttctctt

SEQ ID NO: 8 - DNA, 20 bp, S. aureus
gaaagccacggctaactacg

SEQ ID NO: 9 - DNA, 20 bp, S. aureus
catttcaccgctacacatgg

SEQ ID NO: 10 - DNA, 20 bp, S. aureus
gcaagcgttatccggaatta

SEQ ID NO: 11 - DNA, 20 bp, L monocytogenes
cccttatgacctgggctaca

SEQ ID NO: 12 - DNA, 20 bp, L monocytogenes
cctaccgacttcgggtgtta

SEQ ID NO: 13 - DNA, 20 bp, L monocytogenes
tgcaactcgcctacatgaag

SEQ ID NO: 14 - DNA, 20 bp, Cryptosporidium parvum
atggccgttcttagttggtg

SEQ ID NO: 15 - DNA, 20 bp, Cryptosporidium parvum
ccatttccttcgaaacagga

SEQ ID NO: 16 - DNA, 20 bp, Cryptosporidium parvum
ttccgttaacgaacgagacc

SEQ ID NO: 17 - DNA, 20 bp, Giardia
gacgggtgaaacaggatgat

SEQ ID NO: 18 - DNA, 20 bp, Giardia
ccgtcaatgccttcaagttt

SEQ ID NO: 19 - DNA, 20 bp, Giardia
agaaggcgatcagacaccac

SEQ ID NO: 20 - DNA, 20 bp, Salmonella spp. 23S
tgcgaaagcaggtcatagtg

SEQ ID NO: 21 - DNA, 20 bp, Salmonella spp. 23S
acctacttcagccccaggat

SEQ ID NO: 22 - DNA, 20 bp, Salmonella spp. 23S
aagagttcatatcgacggcg

SEQ ID NO: 23 - DNA, 20 bp, Salmonella
cagccacactggaactgaga

SEQ ID NO: 24 - DNA, 20 bp, Salmonella
gttagccggtgcttcttctg

SEQ ID NO: 25 - DNA, 20 bp, Salmonella
aggccttcgggttgtaaagt

SEQ ID NO: 26 - DNA, 20 bp, Campylobacter jejuni
gtccccagcaaacaggatta

SEQ ID NO: 27 - DNA, 20 bp, Campylobacter jejuni
ccgaaccgttagcaacaaat

SEQ ID NO: 28 - DNA, 20 bp, Campylobacter jejuni
aacgcattaagtgtaccgcc